(19) United States
(12) Patent Application Publication
(10) Pub. No.: US 2008/0268386 A1
(43) Pub. Date: Oct. 30, 2008

(54) Method for embodying a low flame-spread coil incense

(76) Inventor: Chia Mao Lin, Jhunan Township (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/790,919

(22) Filed: Apr. 30, 2007

(51) Int. Cl. A61L 9/02

(52) U.S. Cl. 431/8

(57) ABSTRACT

A method for embodying a low flame-spread coil incense comprises a plurality of coils, radiuses of which are extended outward coil by coil, connected with each other, and between the coils being provided with a specific radial distance. After lighting the coil incense, its bottom is contactingly placed on a refractory material so as to lower flaming temperature for controlling flame spreading time, thereby greatly reducing the quantity of manufacturing material in the condition of having a specific flaming time. Furthermore, by using some characteristics, such as the specific gravity of tar is greater than that of smoke and the condensing effect of the refractory material, the tar may attachably deposit on the refractory material for effectively lowering the smoke quantity.

### BACKGROUND OF THE INVENTION

1. Field of the Invention

The present invention relates to a method for embodying a low flame-spread coil incense that may control a specific distance between the coils and place the coil incense onto a refractory material.

2. Description of the Prior Arts

With reference to FIG. 1, a prior art coil incense includes a plurality of coils 91, radiuses of which are extended outward coil by coil, connected with and matingly abut against each other so as to obtain a longer flaming time, for example, the coil incense 91 may be controlled to flame for 24 hours. As illustrated in FIG. 2, in practical use, the coil incense is hanged onto a receiving dish 92, and the coils 91 in turn fall from inward to outward such that the coil incense naturally forms a spiral distance for sufficiently burning.

However, such a prior art coil incense still has the following disadvantages:

1. The manufacturing material of the coil incense contains single or compounded new or older incense powders, yet because of the limitations of environmental protection and material source, the material price is becoming higher gradually. Moreover, the coils 91 matingly abut against with each other, therefore the manufacturing material consumes in a large quantity.

2. As shown in FIG. 2, the smoke of the prior art coil incense is so thick that the indoor ceiling, wall and lamps and the like will be smoked. In addition, the smoke has a poor influence on humans' health.

3. As illustrated in FIG. 3, even though the coil incense is hanged onto the receiving dish 92, the coils 91 probably abut against each other due to material recovery, and after a long time burning, the outer rim of the coils 91 may absorb the tar in the smoke, so that the surface area of the coils 91 may not provide enough air to aid burning, hence the inwardmost coil 91 is easily extinguishable.

As for TW Pub. No. 574177, entitled “method of making nonsmoke incense” includes changing manufacturing material to achieve a nonsmoke advantage, however, the incense products made by using such a manufacturing method lack dignity owing to nonsmoke, accordingly their acceptance is not high, and the inwardmost coil is also easily extinguishable. Besides, the production cost and the quantity of manufacturing material may not be effectively diminished.

The present invention has arisen to mitigate and/or obviate the afore-described disadvantages.

### SUMMARY OF THE INVENTION

The primary object of the present invention is to provide a method for embodying a low flame-spread coil incense that may control the coil incense to flame at least 12 hours and lower the flaming spread, thereby greatly reducing the quantity of manufacturing material.

Another object of the present invention is to provide a method for embodying a low flame-spread coil incense that due to the bottom of the coil incense is contactingly placed on the refractory material for gently burning, the specific gravity of tar is mostly greater than that of smoke, and the excellent condensing effect of the refractory material, accordingly the smoke may attach onto the holding plane of the refractory material such that the smoke of the coil incense may be effectively lowered.

The present invention will become more obvious from the following description when taken in connection with the accompanying drawings, which show, for purpose of illustrations only, the preferred embodiment in accordance with the present invention.

### BRIEF DESCRIPTION OF THE DRAWINGS

FIG. 1 is a perspective diagram of a prior art coil incense;

FIG. 2 is a perspective diagram illustrating the prior art coil incense being flaming;

FIG. 3 is a perspective diagram illustrating the inner coil of the prior art coil incense being extinguishable;

FIG. 4 is a perspective diagram of a coil incense according to a first embodiment of the present invention;

FIG. 5 is a perspective diagram illustrating the coil incense of the first embodiment of the present invention being flaming;

FIG. 6 is a partially enlarged sectional diagram illustrating the embodying state of a coil and a refractory material of the first embodiment of the present invention;

FIG. 7 is a perspective diagram illustrating the coil incense according to a second embodiment of the present invention being flaming.

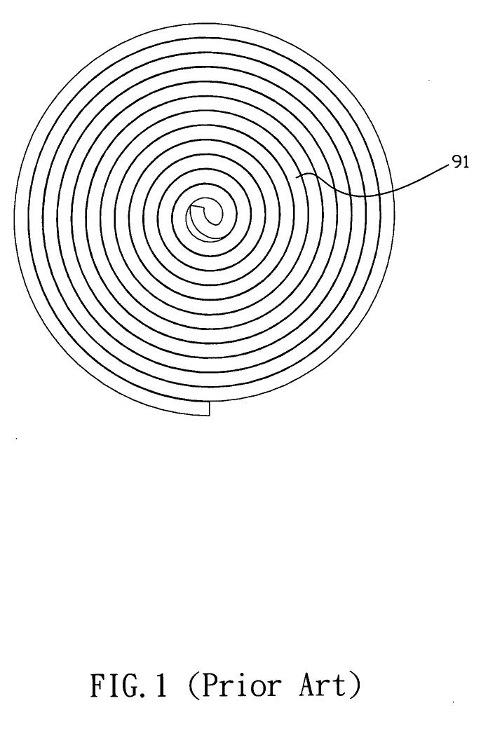

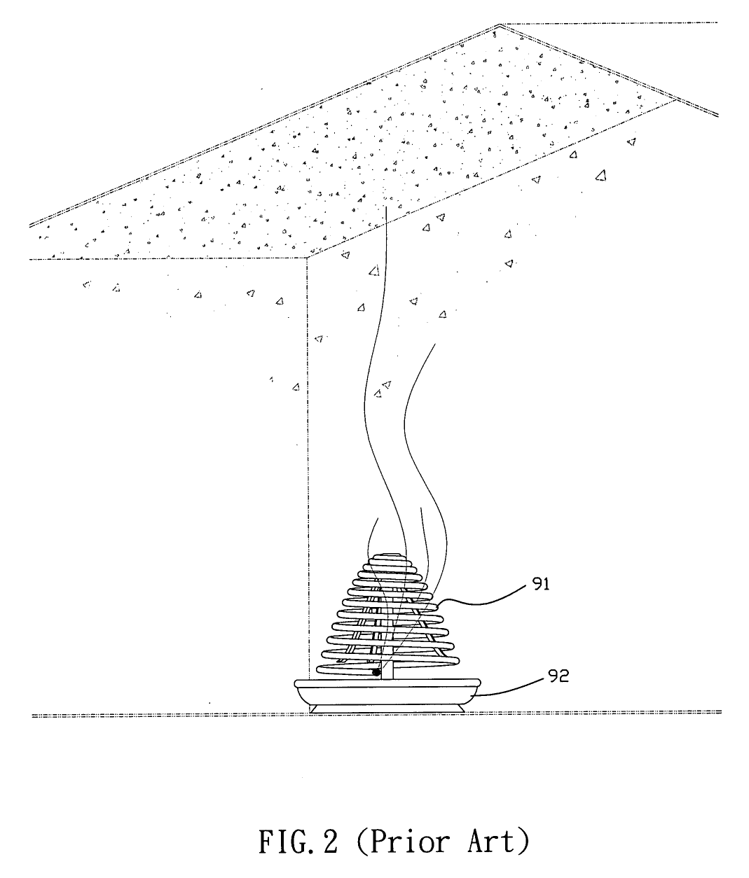

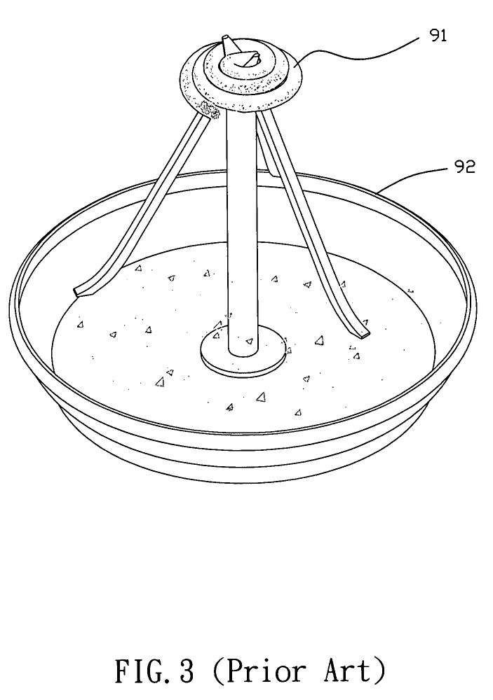

### DETAILED DESCRIPTION OF THE PREFERRED EMBODIMENT

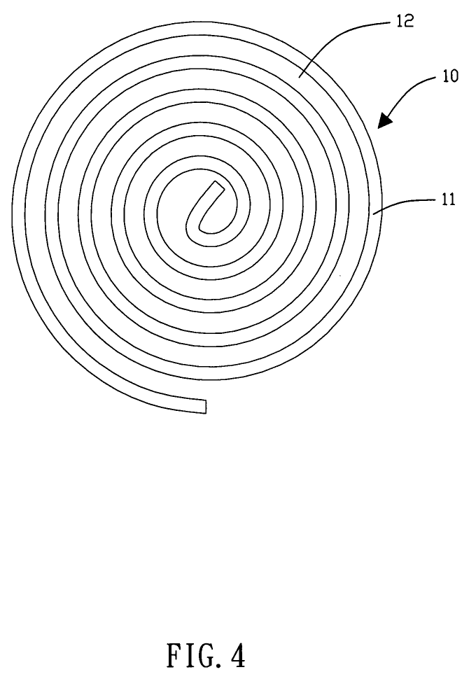

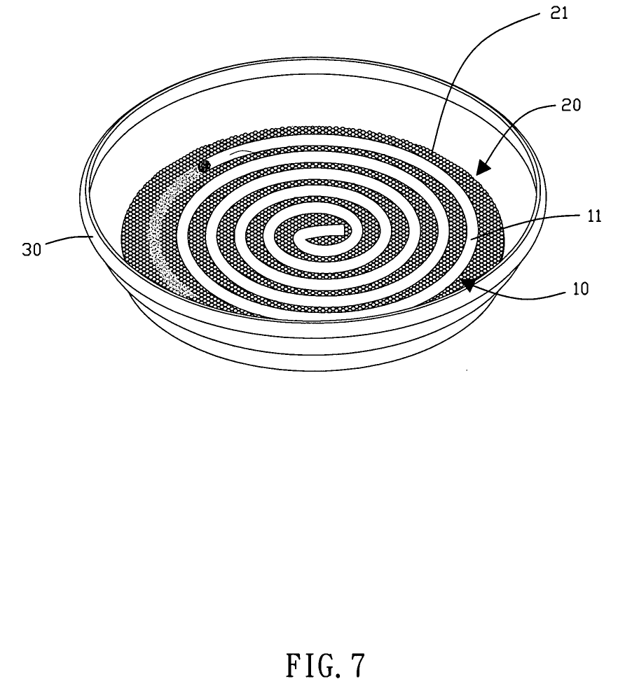

Referring to FIGS. 4-6, a method for embodying a low flame-spread coil incense according to a first embodiment of the present invention comprises a coil incense 10 made of powder materials and formed depended on desired shape, and including a plurality of coils 11, radiuses of which are extended outward coil by coil and cross sections of which are respectively in the shape of a circle, connected with each other, and between the coils 11 being provided with a specific radial distance 12.

A refractory material 20 in the form of a sheet, may be made of undeformable substances, such as quartz, mica sheet, ceramics or calcium (e.g. gypsum or lime) etc, and includes a holding plane 21 formed thereon, for cooperation with the coil incense 10.

In the first embodiment of the present invention, after lighting the coil incense 10, its bottom is contactingly placed on the holding plane 21 of the refractory material 20. Because the cross section of the coil incense 10 is in the shape of a circle, the bottom thereof contacts the refractory material 20 in a linear manner, yet the coil incense 10 is not extinguishable. It is because that the refractory material 10 may utilize its heat resistance and thermal conductivity to reduce flaming temperature, thereby achieving a low flame-spread advantage. For example, in the condition of controlling the flaming time within 12 hours, the flaming length of the coil incense 10 is one half shorter than the prior art coil incense, thereby reducing the quantity of manufacturing material.

Furthermore, due to the bottom of the coil incense 10 is contactingly placed on the refractory material 20 for gently burning, the specific gravity of tar is mostly greater than that of smoke, and the excellent condensing effect of the refractory material 20, accordingly the smoke may attach onto the holding plane 21 of the refractory material 20 such that the smoke of the coil incense 10 may be effectively diminished.

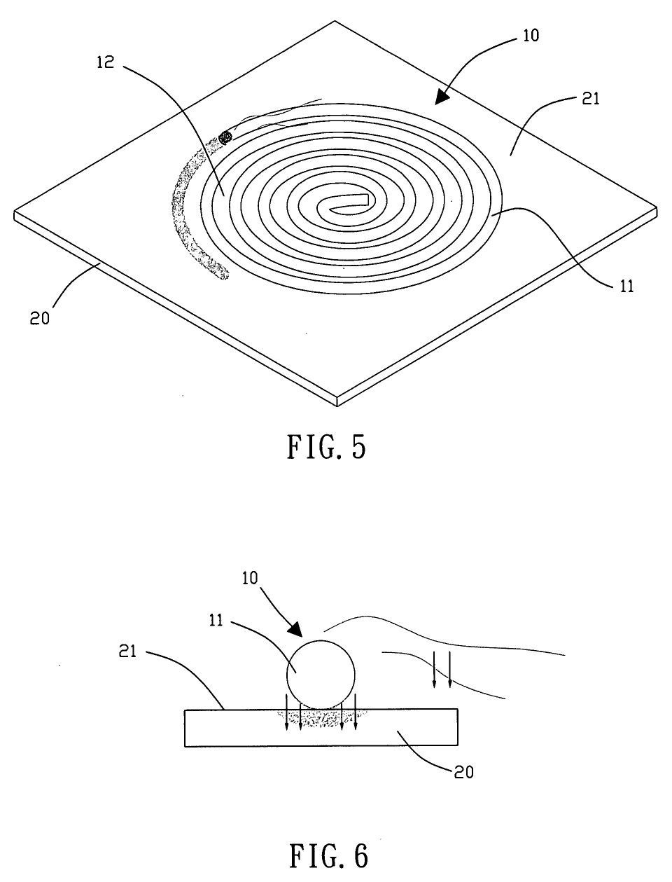

As shown in FIG. 7, a method for embodying a low flame-spread coil incense according to a second embodiment of the present invention comprises applying a receiving dish 30 to receive a refractory material 20 having a plurality of projections received therein and a receiving plane 21 formed in the receiving dish 30. While embodying the second embodiment, the bottom of the coil incense 10 flatly contacts the holding plane 21 of the refractory material 20 after lighting the coil incense 10. Due to the bottom of the coil incense 11 contacts the refractory material 20 in a mostly dot-shaped manner, thus obtaining the lower flame-spread purpose so as to adapt for the large area of the bottom of the coil incense 10, e.g., the polygonal or elliptical cross section. Besides, the surface of the refractory material 20 may be coated Teflon for cleaning the attached tar thereon. Likewise, the Teflon may be coated onto the holding plane 21 as illustrate in FIG. 5.

The invention is not limited to the above embodiment but various modifications thereof may be made. It will be understood by those skilled in the art that various changes in form and detail may made without departing from the scope and spirit of the present invention.

What is claimed is:

1. A method for embodying a low flame-spread coil incense comprising:
a coil incense including a plurality of coils, radiuses of which are extended outward coil by coil, connected with each other, and between said coils being provided with a specific radial distance;
after lighting said coil incense, by lowering the flaming temperature, the flaming time may be controlled up to 12 hours, so as to greatly reduce the quantity of manufacturing material of said coil incense.

2. The method for embodying the low flame-spread coil incense as claimed in claim 1, wherein after lighting said coil incense, it is contactingly placed on said refractory material for continuously flaming and lowering flaming temperature, such that the flame spread may be reduced to control the flaming time, yet said refractory material may be made of heat resistant and undeformable substances, hence the tar of smoke may attach onto said refractory material so as to lower smoke amount.

3. The method for embodying the low flame-spread coil incense as claimed in claim 2, wherein said refractory material in the form of a sheet, includes a holding plane formed thereon for contacting with the bottom of said coil incense to flame in a linear manner.

4. The method for embodying the low flame-spread coil incense as claimed in claim 2, wherein the bottom of said coil incense contacts said refractory material in a mostly dot-shaped manner.

5. The method for embodying the low flame-spread coil incense as claimed in claim 4, wherein said refractory material has a plurality of projections received therein and a receiving plane formed thereon for contacting with the bottom of said coil incense in a mostly dot-shaped manner.

6. The method for embodying the low flame-spread coil incense as claimed in claim 3 or 5, wherein said holding plane of said refractory material is coated with Teflon thereon.

7. The method for embodying a low flame-spread coil incense as claimed in claim 2, wherein said refractory material is made of quartz substance.

8. The method for embodying a low flame-spread coil incense as claimed in claim 2, wherein said refractory material is made of mica sheet.

9. The method for embodying a low flame-spread coil incense as claimed in claim 2, wherein said refractory material is made of ceramics substance.

10. The method for embodying a low flame-spread coil incense as claimed in claim 2, wherein said refractory material is made of calcium substance.
